(19) United States
(12) Patent Application Publication
(10) Pub. No.: US 2018/0344666 A1
(43) Pub. Date: Dec. 6, 2018

(54) VITAMIN K-ENRICHED LIPID EMULSION FORMULATIONS FOR THE TREATMENT OF PHARMA TOXICITY

(71) Applicant: RESQ PHARMA INC., Chicago, IL (US)

(72) Inventors: Guy WEINBERG, Chicago, IL (US); Israel RUBENSTEIN, Chicago, IL (US)

(21) Appl. No.: 15/775,452

(22) PCT Filed: Nov. 11, 2016
(86) PCT No.: PCT/US2016/061707

§ 371 Date: May 11, 2018

Related U.S. Application Data
(60) Provisional application No. 62/255,376, filed on Nov. 13, 2015.

(51) Int. Cl. A61K 31/122; A61K 9/107; A61K 9/00; A61K 47/24; A61K 47/44; A61P 9/00

(52) U.S. Cl. CPC A61K 31/122; A61K 9/107; A61P 9/00; A61K 47/24; A61K 47/44; A61K 9/0019

(57) ABSTRACT

The present disclosure provides a lipid emulsion formulation comprising vitamin K, and methods of treating pharma toxicity.

### CROSS-REFERENCE TO RELATED APPLICATIONS

This application is a PCT international patent application which claims the benefit of U.S. provisional patent application Ser. No. 62/255,376, filed Nov. 13, 2015, the disclosure of which is expressly incorporated by reference.

### FIELD OF THE INVENTION

This disclosure relates to medicine and pharmacology. More specifically, this disclosure relates to Vitamin K-containing lipid emulsion formulations and to methods for treating systemic toxicity using such formulations caused by foreign agents present in the circulation of a mammal by administrating such formulations.

### BACKGROUND

Toxicity from illicit drugs, prescription medication overdoses, or exposure to hazardous chemicals is a major global health concern. Drug overdoses account for nearly 4.6 million emergency department visits and over 80,000 preventable deaths in the United States each year. Overdoses may be treated by specific antidotes including naloxone and flumazenil, but it is difficult and expensive to develop antidotes for each of the commonly overdosed drugs. Gastric lavage and charcoal administration can minimize absorption into the blood-stream, but these techniques are only useful to limit enteral absorption and remain controversial. Gastric lavage has several risks including, perforation of the GI tract or pharynx, aspiration pneumonia, hypoxia, or dehydration and is not effective more than an hour after toxicant ingestion. Charcoal administration also becomes less effective more than an hour after ingestion and is contraindicated for acidic or alkali toxicants. In many cases, the most common approach is to provide the support needed to maintain the patient's stability and relying on the endogenous ability to metabolize or excrete the toxicant.

Several parenteral therapies are being investigated to alleviate or reverse the effects of overdose or toxicity. The general strategy involves injection of colloidal solutions which exhibit a high partitioning of the drug or toxicant. These colloids are thought to create a separate inert pharmacokinetic compartment which reduces the drug or toxicant concentration in free blood and affected tissues to help restore organ function and allow endogenous metabolism, excretion, or redistribution to occur more rapidly. Emulsions and liposomes are the most common colloids investigated for toxicity therapy, but these therapies are still under investigation and are strongly debated in literature.

Intravenous lipid emulsions (ILEs) using the soybean oil-base formulation Intralipid® have been used for treating overdoses of local anesthetics. ILE's are nanometer-sized droplets of triglyceride oils in water stabilized by phospholipid surfactants. ILE usage still remains very controversial for detoxification from other drugs, particularly when the drug is being absorbed by the enteral route.

In addition, lipid resuscitation therapy (LRT) is used clinically as an antidote for local anesthetic systemic toxicity and nonspecific xenobiotic overdose. Fettiplace, M. R. & Weinberg, G. Past, Present, and Future of Lipid Resuscitation Therapy. JPEN J Parenter Enteral Nutr 39, 72S-83S (2015).

Thus, what is needed are better formulations and improved methods for treating systemic toxicity.

### SUMMARY

It has been discovered that certain lipid emulsions containing high concentrations of vitamin K are useful for treating pharma toxicity due to foreign lipophilic or amphiphilic substances, or their metabolites, in the bloodstream of a mammal. This discovery has been explored to develop the present disclosure, which, provides lipid emulsion formulations and methods of using such formulations to treat pharma toxicity.

In one aspect, the disclosure provides lipid emulsion formulation comprising a lipid, vitamin K, an emulsifier, a tonicity modifier, and water.

In some embodiments, vitamin K comprises phylloquinone, menaquinone-4, menaquinone-7, and/or analogs, homologs, mimetics thereof, and/or combinations thereof. In certain embodiments, the amount of vitamin K in the lipid emulsion is greater than 200 μg per kilogram of human or animal body weight.

In certain embodiments, the method comprises administering the lipid emulsion formulation of the disclosure to the mammal, wherein said lipid emulsion comprises vitamin K, about 10 percent to about 40 percent oil by weight, about 1 percent to about 5 percent emulsifier by weight, about 1 percent to about 5 percent tonicity modifier by weight, and about 58 percent to about 88 percent water by weight.

In some embodiments, the lipid in the formulation is a fatty acid, a glycerolipid, a glycerolphospholipid, a sphingolipid, a saccharolipid, a polyketide, a sterol, and/or a prenol. In specific embodiments, the fatty acid is a phospholipid. In certain embodiments, the lipid is an oil. In specific embodiments, the oil is a naturally occurring vegetable or animal oil, a mineral oil, or a chemically-synthesized oil. In some embodiments, the oil comprises soybean oil, avocado oil, flaxseed oil, coconut oil, cottonseed oil, squalene oil, groundnut oil, olive oil, canola oil, corn oil, rapeseed oil, safflower oil, and sunflower oil), animal oils (e.g., fish oil), flavor oil, water insoluble vitamins, mineral oil, and mixtures thereof. In one particular embodiment, the oil comprises soybean oil.

The emulsifier may be natural, semi-synthetic, or synthetic. In some embodiments, the emulsifier comprise lecithin, such as a synthetic lecithin. In particular embodiments, emulsifier comprises dihexanoyl-L-α-lecithin and/or a phospholipid. In specific embodiments, the phospholipid comprises phosphatidylcholine, phosphatidylethanolamine, phosphatidylserine, phosphatidylinositol, phosphatidylglycerol, phosphatidic acid, lysophospholipids, egg or soybean phospholipid and combinations thereof.

In some embodiments, the tonicity modifier comprises glycerin, sorbitol, polyoxyethylated hydrocarbons, C6-C20 saturated aliphatic acids, unsaturated aliphatic acids, and/or mixtures thereof.

In certain embodiments, the lipid emulsion formulation further comprising a co-solvent. In particular, embodiments, the co-solvent comprises an alcohol. In specific embodiments, the alcohol comprises isopropanol, benzyl alcohol, or combinations thereof.

In some embodiments, the lipid emulsion formulation, further comprising a polyethylene glycol (PEG) moiety

In other embodiments, the formulation further comprising a bacteriostat or preservative.

In addition, in some embodiments, the formulation further comprising a biologically active compound which renders the toxic agent non-toxic or which counters the physiological effects of the toxic agent. In particular embodiments, the biologically active ingredient comprises an inotrope. In specific embodiments, the inotrope comprises insulin or levosimendan.

In some embodiments, the formulation may further comprising an adsorbent, such as charcoal, silica gel, and/or mixtures thereof. In particular embodiments, the lipid emulsion formulation comprising particles of about 0.25 μm to about 0.75 μm in diameter.

In another aspect, the present disclosure provides a method of treating pharma toxicity in a mammal, comprising administering to the mammal a therapeutically effective amount of the lipid emulsion formulation of the present disclosure.

In some embodiments, the pharma toxicity is caused by a toxic foreign substance in the bloodstream of the mammal, wherein the toxic foreign substance comprises anesthetics, illicit drugs, cathinones, herbal extracts, herbal supplements, legal drugs, organophosphates, insecticides, herbicides, fungicides, and/or blistering agents. In certain embodiments, the toxic foreign substance is unknown or suspected to be present in the mammal at the time of administration.

In some embodiments, the lipid emulsion formulation is administered intravenously, intra-arterially, intraperitoneally, intralymphatically, intraosseously, rectally, intratechally, inhaled/intratracheally, intraocularly, or topically.

In particular embodiments, the lipid emulsion formulation is intravenously administered at a steady rate of about 0.25 ml to about 0.5 ml per kilogram of the mammal's weight per minute for a time period of about 30 minutes to about 60 minutes. In other embodiments, an initial bolus of the lipid emulsion formulation of between about 1.0 ml to about 3.0 ml per kilogram of the patient's weight is administered to the mammal for a period of about 30 seconds to about 60 seconds.

In a specific embodiments, the mammal to which the lipid emulsion formulation is administered in asystole.

In some embodiments, the lipid emulsion formulation has been dehydrated or freeze-dried to form a solid phase at temperature of about 25° C., and which is rehydrated before administration.

In yet other embodiments, the lipid emulsion formulation is administered to the mammal via a pre-filled delivery device.

In another aspect, the disclosure provides a kit comprising the lipid emulsion formulation and a delivery device.

In yet another aspect, the disclosure is directed to methods for treating toxicity, resulting from cardiovascular and/or neurological impairment, due to foreign lipophilic and amphiphilic substances.

In another aspect, the disclosure provides a method of treating toxicity resulting from foreign substances in the blood of a mammalian subject. In this method, a mammal having toxic levels of pharmaceutical drugs or other toxic substances is intravenously infused with a lipid emulsion formulation such that the toxic substance permeates the emulsion and is redistributed according to its lipid: aqueous partition coefficient onto the surface of the oil droplets and into the non-aqueous (lipid) phase of the emulsion. The lipid particles are typically several hundred nanometers in diameter and therefore a bound drug cannot pass through the endothelial gaps (approximately 4 nanometers) and is trapped in the blood stream thereby decreasing the bioavailability of the toxic substance. Such lipid sinks have wide applicability to the treatment of toxicity associated with lipophilic and amphiphilic substances.

### DESCRIPTION

Throughout this application, various patents, patent applications, and publications are referenced. The disclosures of these patents, patent applications, and publications are hereby incorporated by reference in their entirety into this application in order to more fully describe the state of the art as known to those skilled therein as of the date of the invention described and claimed herein. The instant disclosure will govern in the instance that there is any inconsistency between the patents, patent applications, and publications and this disclosure.

The present disclosure relates to lipid emulsion formulations containing vitamin K and to methods for reducing pharma toxicity or the bioavailability and toxicity of foreign substances present in the circulation of a subject by treatment with such formulations. Conditions caused by a lipophilic or amphiphilic foreign substance, can be prevented and even reversed through an infusion of a lipid emulsion formulation of the present disclosure.

As used herein, “toxicity”, “pharma toxicity”, and “systemic toxicity” refer to severe adverse effects a foreign substance has on the body or a transplanted organ. Such toxicity can occur, e.g., during liver, kidney, or heart transplant where clamps are used to restrict blood flow, seizures, induction of coma, neurological damage and cardiovascular impairment. The adverse effects include, but is not limited, to low blood pressure (hypotension), ischemia reperfusion injury, such as to the heart after a heart attack, cardiac arrhythmia, cardiotoxicity, cardiovascular collapse, cardiac arrest, heart failure, asystole, or stroke, such as to the brain. Neurological impairments and symptoms, such as obtundation, agitation, coma and seizures, are typical of local anesthetic toxicity (and toxicity caused by similar substances) and generally, although not always, precede cardiac symptoms and effects. More particularly, ischemia is a restriction in blood supply or circulation, generally due to conditions in the blood vessels, hypotension, or low cardiac output, with resultant damage or dysfunction of tissue. Prolonged ischemia can also cause neurological damage. “Cardiotoxicity” refers to impaired automaticity and propagation of electrical impulses through the heart and its conducting system as well as damage to heart muscles and failure by the heart to adequately pump blood through the body as a result of the toxin. This can cause dangerously low blood pressure and cardiac output and therefore ischemia of important organs including the nervous system and heart, “Asystole” is a state of no contractions of the heart with no cardiac output or blood flow. In asystole, the heart will not typically respond to defibrillation because it is already depolarized, making resuscitation of the patient extremely difficult. Asystole is one of the conditions required for a medical practitioner to certify death.

The lipid emulsion formulations of the disclosure comprise vitamin K, a lipid, an emulsifier, a tonicity modifier, and water. Additional ingredients may include a surfactant, a co-solvent, a bacteriostat, a preservative, a biologically active ingredient, and/or an adsorbent

Lipids may be broadly defined as hydrophobic or amphiphilic small molecules; the amphiphilic nature of some lipids allows them to form structures such as vesicles, multilamellar or unilamellar liposomes, or membranes in an aqueous environment. Lipids useful in the formulation can be naturally occurring, synthetic or chemically synthesized, or semi-synthetic (modified materially occurring lipids). Naturally occurring lipids useful in the formulation include molecules such as fats, waxes, sterols, fat-soluble vitamins (such as vitamins A, D, E, and K), monoglycerides, diglycerides, triglycerides, phospholipids, and the like. Naturally occurring lipids may be divided into eight categories: fatty acids, glycerolipids, glycerophospholipids, sphingolipids, saccharolipids, and polyketides; sterols; and prenols. Also useful are derivatives of fatty acids (including tri-, di-, monoglycerides, and phospholipids), as well as sterol-containing metabolites such as cholesterol.

Useful Phospholipids may be any naturally occurring or synthetic phospholipid, whether saturated or unsaturated. They may be in any form, including salted or desalted, hydrogenated or partially hydrogenated, or natural, semisynthetic (modified) or synthetic. Useful phospholipids include, but are not limited to, phosphatidylcholine, phosphatidylethanolamine, phosphatidylserine, phosphatidylinositol, phosphatidylglycerol, phosphatidic acid, lysophospholipids, egg or soybean phospholipid and combinations thereof.

Useful lipids for the emulsion also include oils such as any oil that can be absorbed and metabolized by the mammalian body and which do not elicit toxic side effects such as a naturally occurring vegetable or animal oil, a mineral oil, or a chemically-synthesized oil. Useful oils include monoglycerides, diglycerides, triglycerides, and/or mixtures thereof. Useful plant oils include, but are not limited to, soybean oil, cottonseed oil, safflower oil, corn oil, coconut oil, sesame oil, peanut oil, olive oil, avocado oil, flaxseed oil, cottonseed oil, squalene oil, ground-nut oil, olive oil, canola oil, rapeseed oil, safflower oil, sunflower oil and a mixtures thereof. One useful oil is soybean oil. In addition, the oil can be an animal oil or a fish oil such as cod liver oil. The oil can alternatively be a mineral oil or a chemically-synthesized oil such as 2-linoleoyl-1,3-dioctanoyl glycerol. Semisynthetic mono-, di- or triglycerides, and mixtures thereof, may also be used and include Medialipid™ (a mixed medium and long-chain triglyceride emulsion from B. Braun Melsunsen AG, Germany), rac-glyceryl-1-monopalmitic, acyl glyceryl-1-monoolein, 1,2-dipalmitic, 1,3-dipalmitic, trimyristin, tripalmitin, tristearin, triolein, trilaiden and the like.

As described above, the lipid emulsion formulations according to the disclosure include vitamin K. Chemically, the vitamin K family comprises 2-methyl-1,4-naphthoquinone (3-) derivatives. Vitamin K has two natural vitamers: vitamin K1 and vitamin K2. Vitamin K1, also known as phylloquinone, phytomenadione, or phytonadione, is the “plant” form of vitamin K, although it is active as a vitamin in animals. Vitamin K2, is the main storage form in animals. It has several subtypes or homolys which differ in isoprenoid chain length. These vitamin K2 homologs are called “menaquinones” (MK-n) (Menaquinone, vitamin K, n=the number of isoprenoid side chain residues) (see FIG. 1B). Menaquinones are primarily of microbial origin and thus commonly found in fermented foods. However, Menaquinone-4 (abbreviated MK-4), which has four isoprene residues in its side chain (see FIG. 1C), and is also known as “menatetrenone” MK-4 is synthesized in certain animal tissues from vitamin K1.

Vitamin K can be present in very high concentration in the lipid emulsion. In this instance, Vitamin K improves mitochondrial metabolism but in addition to reversing vitamin K depletion that occurs clinically (e.g., in patients on Coumadin, or with liver synthetic deficiency or gastrointestinal malabsorption), thereby reducing the possibility of life-threatening hemorrhage. There is no known toxicity associated with high doses (dietary or supplemental) of the phylloquinone (vitamin K1) or menaquinone (vitamin K2) forms of vitamin K.

The emulsifier in the lipid emulsion formulation may be in any form, including salted or desalted, hydrogenated or partially hydrogenated, or natural, semisynthetic (modified) or synthetic. It may be a naturally-occurring phospholipid such as those derived from egg or soy sources. Exemplary phospholipids include, but are not limited to, egg yolk phospholipids, hydrogenated egg yolk phospholipids, soybean phospholipids, hydrogenated soybean phospholipids, and mixtures thereof. For example, the phospholipid is egg yolk phospholipid. The emulsifier also can be a synthetic lecithin such as dihexanoyl-L-α.-lecithin. Among other emulsifiers useful in the emulsion of the present disclosure are other glycerophospholipids such as phosphatidylcholine, cholesterol, stearylamine, phosphatidylserine, phosphatidylglycerol, and other lipids.

The tonicity modifier can be glycerin, sorbitol, polyoxyethylated hydrocarbons, and C6-C20 saturated or unsaturated aliphatic acids.

The optional co-solvent can be an alcohol such as isopropanol, benzyl alcohol, and the like. The percent weight of water is decreased by the equivalent percent weight of co-solvent.

The optional bacteriostat or preservative can be any commercially available stabilizer which are non-toxic.

The optional adsorbent can be, for example, charcoal, silica gel, and the like.

The biologically active ingredient can be any useful drug or reactant which can render the toxic agent non-toxic or which may act to counter the physiological effects of the toxic agent. For example, the biologically active ingredient can be an inotrope, which is an agent that alters the force or energy of muscular contractions, such as insulin or levosimendan.

The lipid emulsion formulations can take various forms, depending on the lipid used in the formulation. For example, the lipid emulsion formulation can take lamellar, non-lamellar, liquid crystalline, or L3 phase forms, or combinations thereof.

As discussed above, a bulk non-lamellar phase is typically a thermodynamically stable system. In addition, this bulk phase may be dispersed in a polar or nonpolar solvent to form particles of a non-lamellar (especially liquid crystalline) phase in a bulk solvent. This allows the advantages of bulk non-lamellar phases to be applied in situations where use of a bulk non-miscible phase would cause problems, such as in parenteral applications. Further control of a compound's release profile may also be achieved by such a dispersion of non-lamellar particles.

Liquid crystalline or L3 phase can be in or near thermodynamic equilibrium with the excess solvent and may be dispersed into colloidally stable dispersions of non-lamellar particles. Such particles may be fully (i.e. thermodynamically) stable, or may gradually degrade, thereby providing control over the release profile for active agents formulated therewith. The formation of dispersions can be spontaneous or as the result of mechanical force such as shearing or ultrasound. These non-lamellar particles are of considerable interest in the delivery of active agents and have been proposed as carriers for many such actives. Dispersions containing active ingredients and particularly those for intravenous administration to the human or animal body are desirably colloidal, that is they should be of a particle size no greater than 10 μm, especially no greater than 5 μm and particularly no greater than 1 μm. If particles within the dispersion exceed this size then the dispersion may not be colloidally stable and there is a considerable risk of causing embolism when the preparation is administered intravenously. Furthermore, it is desirable that the distribution of particle sizes be narrow to maximize control over the release of any active agent. Where a particulate composition is to be administered by a method other than intravenously (e.g. orally, intramuscularly, subcutaneously, rectally or by inhalation), then the particles need not necessarily be colloidal but it remains advantageous to provide a well characterized and reproducible particle size distribution in order to control the rate of decomposition of the particles and/or release of the active agents.

In formulating the emulsion, vitamin K is in the range of about 10 μg per kilogram of patient body weight or greater. For example, vitamin K can be present in the formulation at about 10 μg/kg patient body weight to about 1,000 μg/kg patient body weight, or greater than about 1,000 μg/kg patient body weight. The lipid component is present in the formulation in the range of about 10 percent to about 30 percent by weight of the composition, or between about 20 percent and 30 percent, or about 30 percent. The emulsifier in the formulation is present in an amount of about 1 percent to about 5 percent by weight of the composition or lesser or greater. The tonicity modifier in the formulation is present in an amount of about 1 percent to about 5 percent by weight of the composition or lesser or greater. Water is present in the formulation in the range of about 68 percent to about 88 percent by weight or lesser or greater.

The disclosure also provides methods of treating pharma toxicity in a mammal caused by one or more foreign substances already administered to or ingested by the mammal. In this method, the lipid emulsion formulation is administered to the mammal by intravenous, intraarterial, intraperitoneal, intralymphatic, intraosseous, rectal, intratechal, inhaled/intratracheal, intraocular, and topical (for example in the eye). One useful route of administration is by intravenous administration.

One method for the treatment of systemic toxicity according to the disclosure comprises causing a patient in need of such therapy rapidly to become lipemic by the intravenous infusion of an initial large bolus dose of the emulsion formulation, followed by a slower, steady-state rate of infusion of the emulsion formulation. Although the rate of infusion can vary with respect to the particular emulsion utilized with the toxic agent involved and with the particular patient, by way of example, an initial rate of the infusion may be in the range of about 1.5 m I/kg, over a time period of about 30 to 60 seconds, followed by a steady-state rate in the range of about 0.25 ml/kg/min to about 0.5 ml/kg/min for a time period of about 30 minutes.

The foreign toxic substance in the bloodstream of the mammal in need of treatment with the lipid emulsion formulations of the disclosure may be lipophilic or amphipathic. Non-limiting examples of such toxic substances include (1) legal drugs such as GABAergic medications, benzodiazerpine, diphenhydramine, antipsychotic medications, antidepressant medications, β-blockers, calcium channel blockers, or cancer drugs (e.g., Adriamycin, doxorubicin); (2) illegal drugs such as cocaine, THC, ‘bath salts’ and other cathinones; (3) herbal extracts or supplements, including cannabinoids; (4) anesthetics such as bupivacaine, lidocaine, mepivacaine, etidocaine, amethocaine, tetracaine, procaine, 2-chloroprocaine, prilocaine, procainamide, levobupivacaine, ropivacaine, dibucaine; (5) organophospates, including those considered as possible bio-terror (and select) agents, (e.g., soman, sarin, malathion, parathion, diazinon, etc.); (6) insecticides such as ivermectin, permethrin; (7) herbicides, including glycophosphate; (8) fungicides; (9) blistering agents like sulfur and nitrogen mustard; and (10) any combinations thereof. The foreign substance may also be unknown.

While not being limited by any mechanism, the lipid emulsion formulation infused into the bloodstream of the mammal may achieve this by absorbing the foreign substance, or its metabolites, from the bloodstream, thereby reducing its bioavailability. For example, the cardiac and/or neurological impairments resulting from elevated plasma levels of the foreign substance alleviation are reduced by virtue of reducing the effective, or non-lipid bound, concentrations of the foreign substance.

The lipid emulsion formulation of the present disclosure can be used to specifically threat foreign substances which cause cardiovascular impairment or neurological impairment. “Treating a cardiovascular or neurological impairment”, such as cardiotoxicity, coma or ischemia of the brain or heart, refers to decreasing, eliminating and in some cases reversing the specific adverse cardiovascular or neurological effects a foreign substance has on the body. For example, where a foreign toxin results in low cardiac output and ischemia of the brain or heart, administration of the present lipid emulsion formulation removes the foreign substance from the bloodstream thereby restoring at least a portion of the cardiac output and lessening the ischemia of the brain or heart.

### EQUIVALENTS

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

1. A method of treating cardiac pharma toxicity in a patient caused by one or more foreign substances already administered to or ingested by the patient, said method comprising the steps of:
intravenously administering a lipid emulsion composition to the patient wherein the lipid emulsion comprises between about 1.0 and about 5.0 percent vitamin K by weight, about 10 and about 40 percent oil by weight, about 1 to about 5 percent emulsifier by weight, about 1 to about 5 percent tonicity modifier by weight, about 58 to about 88 percent water by weight, and an inotrope,
wherein the one or more foreign substances are lipophilic or amphiphilic substances able to be absorbed by the lipid emulsion composition in the patient's bloodstream.

2. The method of claim 1 wherein vitamin K is selected from the group consisting of phylloquinone, menaquinone-4, and menaquinone-7.

3. (canceled)

4. The method of claim 1 wherein the oil is a naturally occurring vegetable or animal oil, a mineral oil, or a chemically-synthesized oil.

5. The method of claim 1 wherein the oil is selected from the group consisting of soybean oil, avocado oil, flaxseed oil, coconut oil, cottonseed oil, squalene oil, groundnut oil, olive oil, canola oil, corn oil, rapeseed oil, safflower oil, and sunflower oil, animal oils, fish oil, flavor oil, water insoluble vitamins, mineral oil, and mixtures thereof.

6. (canceled)

7. The method of claim 1 wherein the emulsifier is a synthetic lecithin, such as dihexanoyl-L-α.-lecithin, or a phospholipid such as phosphatidylcholine, phosphatidylethanolamine, phosphatidylserine, phosphatidylinositol, phosphatidylglycerol, phosphatidic acid, lysophospholipids, egg or soybean phospholipid or combinations thereof and the emulsifier may be in any form, including salted or desalted, hydrogenated or partially hydrogenated, or natural, modified, semisynthetic or synthetic.

8. The method of claim 1 wherein the tonicity modifier is selected from the group consisting of glycerin, sorbital, polyoxyethylated hydrocarbons, and C₆-C₂₀ saturated and unsaturated aliphatic acids.

9. The method of claim 1 wherein an alcohol is used as a co-solvent.

10. The method of claim 1 wherein the lipid emulsion composition further comprises a bacteriostat, a preservative, an adsorbent, or any combination thereof.

11. The method of claim 1 wherein the biologically active ingredient is a desired drug or reactant which can render the toxic agent non-toxic or which may act to counter the physiological effects of the toxic agent.

12. (canceled)

13. The method of claim 1, wherein the inotrope is selected from the group consisting of: insulin, levosimendan, digoxin, dopamine, dobutamine, norepinephrine, milrinone, omecamtiv mecarbil, amiodarone, berberine, calcium, catecholamines, dopexamine, epinephrine, isoprenaline, angiotensin II, eicosanoids, prostaglandins, phosphodiesterase inhibitors, enoximone, milrinone, amrinone, theophylline, and glucagon.

14. (canceled)

15. The method of claim 1 wherein foreign substances are selected from the group consisting of anesthetics, illicit drugs, cathinones, herbal extracts, legal drugs, organophosphates, insecticides, herbicides, fungicides, and blistering agents.

16. The method of claim 1 wherein the foreign substance is unknown or suspected to be present at the time of intravenous administration.

17. The method of claim 1 wherein the lipid emulsion composition is administered at a steady rate of about 0.25 ml to about 0.5 ml per kilogram of the patient's weight per minute for a time period of about 30 minutes to about 60 minutes.

18. The method of claim 1 wherein an initial bolus of the lipid emulsion composition between about 1.0 ml to about 3.0 ml per kilogram of the patient's weight is administered to the patient for a period of about 30 seconds to about 60 seconds.

19. (canceled)

20. The method of claim 1 wherein the patient receiving the lipid emulsion composition is in asystole.

21. The method of claim 1 wherein the lipid emulsion is used to treat a poisoned animal.

22. A lipid emulsion formulation comprising:
about 1.0 to about 5.0% per kilogram of patient body weight vitamin K;
a lipid;
an emulsifier;
a toxicity modifier;
a co-solvent;
a preservative;
an adsorbent, and
an inotrope.

23. The method of claim 1, wherein the inotrope is positive and stimulates and increases the force of contractions.

24. The method of claim 1, wherein the inotrope is negative and weakens the force of muscular contractions.

25. The method of claim 24, wherein the negative inotrope is selected from the group consisting of: beta blockers, non-dihydropyridine calcium channel blockers, diltiazem, verapamil, clevidipine, class IA antiarrhythmics, quinidine, procainamide, disopyramide, class IC antiarrhythmics, and flecainide.
